(19) United States
(12) Patent Application Publication
(10) Pub. No.: US 2020/0147285 A1
(43) Pub. Date: May 14, 2020

(54) HEART SUPPORT SYSTEM, CANNULA ARRANGEMENT FOR A HEART SUPPORT SYSTEM AND USE OF THE HEART SUPPORT SYSTEM

(71) Applicant: Rheinisch-Westfälische Technische Hochschule (RWTH) Aachen, Aachen (DE)

(72) Inventors: Jonas Gesenhues, Aachen (DE); Dirk Abel, Aachen (DE)

(21) Appl. No.: 16/620,304

(22) PCT Filed: Jun. 4, 2018
(86) PCT No.: PCT/EP2018/064617

§ 371 Date: Dec. 6, 2019

(30) Foreign Application Priority Data
Jun. 6, 2017 (DE) 10 2017 112 437.3

(51) Int. Cl. A61M 1/12; A61M 1/10

(52) U.S. Cl. CPC A61M 1/122; A61M 1/1008; A61M 2205/3334; A61M 1/1081; A61M 1/1086

(57) ABSTRACT

The disclosure relates to a heart support system with at least one system unit that includes a pump arrangement, a first connection apparatus connected to the pump arrangement and a second connection apparatus connected to the pump arrangement, wherein the pump arrangement has a first pump that is fluidically connected in a first flow path between the first and the second connection apparatus. The pump arrangement further has an intermediate reservoir and a second pump, which are connected in series together with the first pump in the first flow path, wherein the intermediate reservoir is fluidically arranged between the first pump and the second pump. The disclosure further relates to a cannula arrangement for a heart support system of this kind and a corresponding use of the heart support system.

### INTRODUCTION

The disclosure relates to a heart support system with at least one system unit, which comprises a pump arrangement, a first connection means connected to the pump arrangement and a second connection means connected to the pump arrangement, wherein the pump arrangement comprises a pump fluidically interconnected in a first flow path between the first and the second connection means.

The disclosure further relates to a cannula arrangement for such a heart support system and a corresponding use of the heart support system.

Such a heart support system is also denoted as ventricular assist device (VAD) in the English language. Such a heart support system can be configured for the left ventricle (LVAD), for the right ventricle (RVAD) as well as for both ventricles (BVAD).

Frequently, the use of such heart support systems focuses on the body's blood supply, not on relieving the heart to enable a therapy of the underlying disease. Usually, the application of a heart support system leads to an inherent increase in the afterload of the heart. This not only increases the load on the heart, but is also accompanied by additional difficulties, for example a permanent retention of the outlet heart valve in the closed state or even a growing together of this heart valve in said closed state.

In the context of the present disclosure, the vessels of a cardiovascular system are basically referred to as blood vessels, irrespective of whether they are part of the heart or of the circulatory system.

Document WO 2014/202051 A1 shows a heart support system with a system unit which comprises a pump arrangement, a first connection means connected to the pump arrangement and a second connection means connected to the pump arrangement. The pump arrangement in this case comprises a pump which is fluidically inter-connected in a first flow path between the first and the second connection means. The heart support system described in this document is usually described as a heart support system for the left ventricle.

### SUMMARY

It is an object of the disclosure to provide measures for heart support, which help in overcoming the difficulties mentioned above.

In the heart support system according to the disclosure with at least one system unit which comprises a pump arrangement, a first connection means connected to the pump arrangement and a second connection means connected to the pump arrangement, wherein the pump arrangement comprises a first pump which is fluidically interconnected in a first flow path between the first and the second connection means, it is provided that the pump arrangement further comprises an intermediate reservoir and a second pump which are connected together with the first pump in a series connection in the first flow path, wherein the intermediate reservoir is fluidically arranged between the first pump and the second pump. The heart support system according to the disclosure enables to relieve an insufficient heart so that on the one hand the blood requirement of the body can be provided by the support system, and on the other hand an inherent additional load on the heart can be avoided or at least significantly suppressed by the heart support system.

The behavior as well as the performance of the heart of humans and other living beings is influenced by the direct hydraulic load, in addition to the blood requirement of the body. A distinction is made between preload and afterload. The preload is the amount of inflowing blood into the ventricles during the relaxation phase which the heart has to overcome. The afterload results from the hydraulic resistance the heart has to cope with during a contraction. These loads exist both for the right and the left ventricle. They are also coupled with each other via the circulatory system.

By means of the measures according to the disclosure, it is possible to set the two different loads which act on the heart, namely the preload and the afterload, independently from each other. To this end, the heart support system comprises one or more system unit(s) for selectively influencing the pressure and volume characteristics in the respective ventricle within the heart cycle. The possibility for this influence is achieved according to the disclosure by the series connection first pump—intermediate reservoir—second pump. In this case, the intermediate reservoir forms an intermediate or buffer storage. Due to the reservoir, the flow rate can be temporarily separated from the pressure conditions—at least to a certain degree—in connection with the pumps. In other words, the two pumps, between which the intermediate reservoir is arranged, can generate different flow rates and/or pressure differences over a certain period of time which enables said selective influence of the pressure and volume characteristics. The connection means are connection means for connection to a respective blood vessel of the corresponding cardiovascular system.

According to an embodiment of the disclosure, it is provided that the system unit comprises a third connection means connected to the pump arrangement, which is fluidically connected to the intermediate reservoir in such a way, that the second pump and the intermediate reservoir are connected in a series connection in a second flow path between the second and the third connection means.

It is advantageously provided, according to an embodiment, that the system unit or by use of a plurality of system units at least one of these system units comprises a cannula arrangement for such a connection of the second and the third connection means to a blood vessel, that the direct blood flow through this blood vessel is blocked completely or at least partially at one point and is bypassed via the second flow path with the series connection of intermediate reservoir and second pump. The cannula arrangement is in particular formed as a blocking cannula arrangement (also called blocking cannula), which has a blocking element for completely blocking the direct blood flow through the blood vessel.

According to a further embodiment of the disclosure in the second flow path between the second and the third connection means a third pump is interconnected so that a series connection of the second and the third pump and the intermediate reservoir fluidically interconnected between these pumps is achieved.

According to an embodiment of the disclosure, the system unit or, in the case of a plurality of system units, at least one of these system units comprises at least one cannula for connecting the first connection means and/or the second connection means to a corresponding blood vessel. Such a cannula is also often referred to as a conventional cannula.

According to a further embodiment of the disclosure, it is provided that the first connection means and the third connection means are connected at the suction side to the pump arrangement and that the second connection means is connected at the pressure side to the pump arrangement.

According to yet another embodiment of the disclosure, the heart support system comprises two system units. Then, one system unit is, for example, provided for the left ventricle and the other system unit for the right ventricle. The heart support system then as a whole is a BVAD.

Here, it is provided that the heart support system comprises a further pump, which is fluidically interconnected between the intermediate reservoirs of the two system units. This measure results in another setting option.

According to a further embodiment of the disclosure, the heart support system comprises a control and/or regulating means for controlling the pump.

According to yet another embodiment of the disclosure, the heart support system further includes pressure sensors for measuring the pressure in blood vessels, flow sensors for measuring the flow velocities of the blood in blood vessels and/or sensors for measuring the filling volume of the ventricles.

In the cannula arrangement according to the disclosure for an aforementioned heart support system it is provided that it is set up for such a connection of the second and the third connection means to a blood vessel such, that the blood flow directly through this blood vessel is blocked completely or at least partially at one point and is bypassed via the second flow path with the series connection of intermediate reservoir and second pump. In particular, the cannula arrangement is formed as a cannula arrangement comprising a blocking member for completely blocking the direct blood flow through the blood vessel.

In the use according to the disclosure, it is provided that the abovementioned heart support system is used for the therapeutic treatment of a heart and/or for the scientific examination of a heart.

### BRIEF DESCRIPTION OF THE FIGURES

The disclosure will now be described by way of example with reference to the accompanying drawings based on example embodiments, wherein the features shown below may represent an aspect of the disclosure both individually and in combination.

In the drawings:

FIG. 1 shows an arrangement with a heart and a heart support system according to a first embodiment of the disclosure;

FIG. 2 shows a heart support system according to a second embodiment of the disclosure; and

FIG. 3 shows an arrangement with a heart and a heart support system according to a third embodiment of the disclosure.

### DETAILED DESCRIPTION

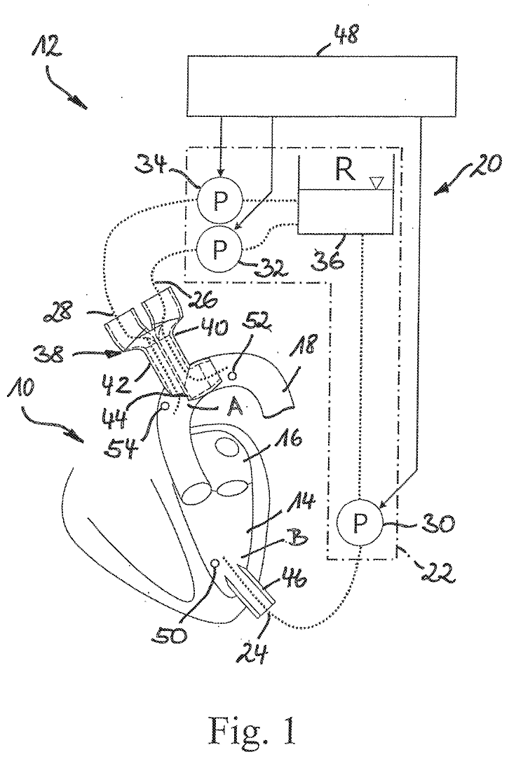

FIG. 1 shows a schematic diagram of a heart 10 and a heart support system 12. In the representation of the heart only a few details of the left half of the heart and its connection to the circulatory system, such as the left ventricle 14, the left atrium 16, and aorta 18, are explicitly shown.

The heart support system 12 shown in FIG. 1 is a system for the left ventricle and therefore comprises only one system unit 20. This system unit 20 includes a pump arrangement 22 and three connection means 24, 26, 28 fluidically connected to the pump arrangement 22. The pump arrangement 22 in turn comprises three pumps 30, 32, 34 and an intermediate reservoir 36. The first of these pumps 30 is fluidically interconnected in a first flow path between the first and the second of the connection means 24, 26, the second and the third of these pumps 32, 34 and the intermediate reservoir 36 are fluidically interconnected in a series connection in second flow path which extends between the third and the second connection means 28, 26. In this series connection, the intermediate reservoir 36 is fluidically interconnected between the second and the third pump 32, 34. As viewed from the reservoir 36 the first connection means 24 is fluidically connected downstream of the first pump 30, the second connection means 26 is fluidically connected downstream of the second pump 32 and the third connection means 28 is fluidically connected downstream of the third pump 34.

The system unit 20 further includes a cannula arrangement 38 of two cannulas 40, 42 for a connection of the second and third connection means 26, 28 to a connection point A in a blood vessel, namely the aorta 18, such that the blood flow directly through this blood vessel 18 is blocked at one point except for very low leakage currents and is bypassed via the second flow path with the series connection of the third pump 34, the intermediate reservoir 36 and the second pump 32. The cannula arrangement 38 comprises a blocking member 44 formed by the walls of the cannulas 40, 42 for completely blocking the direct blood flow through the blood vessel 18. Finally, the system unit 20 moreover comprises a cannula 46 for a connection of the first connection means 24 to a connection point B in another blood vessel, namely the ventricle 14.

The heart support system 12 further comprises a regulating system comprising a control and/or regulating means 48 for controlling the pumps 30, 34, 36 as well as pressure sensors 50, 52, 56 for measuring the pressure in the blood vessels 14, 18. The pressure sensors 50, 52, 54 are signal technically connected to the control and/or regulating means 48 (not shown here). The first pressure sensor 50 measures the pressure in the ventricle 14, the second pressure sensor 52 measures the pressure in the aorta 18 behind the blocking element 44 of the cannula arrangement 38 and the third pressure sensor 54 measures the pressure in the aorta 18 before the blocking element 44 of the cannula arrangement 38.

The system unit 20 of the heart support system 12 is an electromechanical apparatus which is used in combination with the control and/or regulating means 48 for interaction with the cardiovascular system.

The heart support system 12 is for use with the live cardiovascular system of the human or animal. Furthermore, the heart support system 12 enables a systematic research and implementation of a targeted, therapeutic support of the diseased (insufficient) heart 10. Herein, the heart support system 12 enables a targeted and largely isolated manipulation of the cardiac loads, in particular to adjust the two different loads (pre- and afterload), for each ventricle independently of each other.

In use, the heart support system 12 enables to place a load or to relieve the load on a (diseased) heart 10 so that on the one hand the blood requirement of the body can be provided by the system 12, and on the other hand the load on the heart 10 can be selected such that, for example (i) in a therapeutic application a recovery of the diseased heart is optimally promoted. Such a promotion can be understood, for example, as a targeted time-limited load, i.e. a training. On the other hand, (ii) for research purposes a defined load situation of the heart 10 can be simulated by means of this heart assist system 12.

In conventional heart support systems 12, the manipulation of the cardiac loads is untargeted and unintentionally coupled, thus, typically an inherent increase in the after-load is associated with a reduction of the preload, or unwanted side effects occur, such as the lack of the opening of the discharge heart valves (aortic/pulmonary valve), so that the natural functioning of the heart no longer exists. These deficiencies can be solved by the heart support system 12.

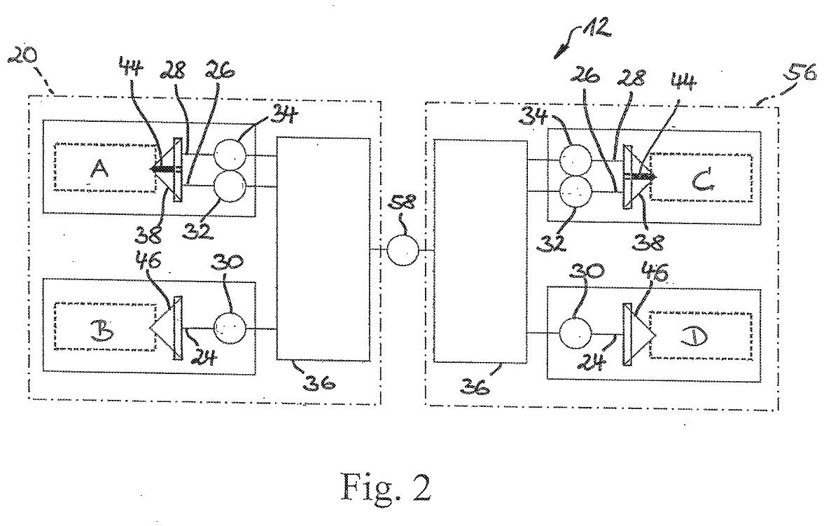

FIG. 2 shows—also in a schematic diagram—another embodiment of the heart support system 12, wherein the regulating components 48, 50, 52, 54 are not shown. The heart support system 12 shown in this figure is a heart support system 12 for both ventricles (BVAD). It therefore has two system units 20, 56 identically in their structure and function. These correspond in their structure and their functionality essentially to the system unit 20 of the heart support system 12 shown in FIG. 1, so that hereinafter the differences are primarily to be discussed.

In order to be able to selectively couple the two system units 20, 56 fluidically to each other, a further pump 58 is fluidically interconnected between the two intermediate reservoirs 36 of the system units 20, 56. This pump 58, too, is selectively controlled via the control and/or regulating means 48. This results in a further degree of freedom in the coupling of pre- and afterload of both ventricles.

FIG. 2 moreover includes an overview of the complete configuration of the heart support system 12 for both ventricles. The components included are described in more detail below.

The connecting or connection points A, B, C and D designated in FIG. 2 are:

A: the aorta 18 or the pulmonary vein;

B: the left ventricle 14, the aorta 18, the left atrium 16 or the pulmonary vein;

C: the right ventricle, the pulmonary artery or one of the vena cava; and

D: the pulmonary artery or one of the vena cava.

There may also be several connection points of one type, for example several connection points of type A.

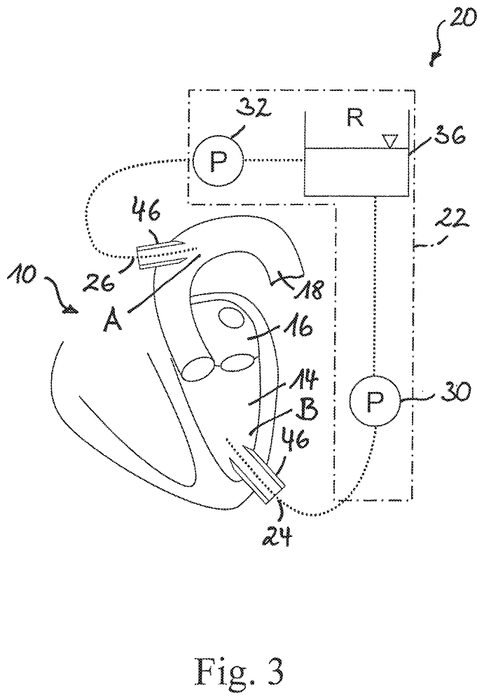

FIG. 3 again shows an arrangement with a heart 10 and with a system unit 20 of a heart support system 12 with a relatively simple structure. The illustration of the heart 10 corresponds to the illustration of FIG. 1. The heart support system 12 shown in FIG. 3, too, is a system for the left ventricle. The system unit 20 comprises the pump arrangement 22 and only two connection means 24, 26 which are fluidically connected to the pump arrangement 22. The pump arrangement 22 in turn also comprises only two pumps 30, 32 and the intermediate reservoir 36. These pumps 30, 32 and the intermediate reservoir 36 are interconnected in a series connection in the first flow path between the two connection means 24, 26. Here, the intermediate reservoir 36 is fluidically disposed between the first pump 30 and the second pump 34. The system unit 20, moreover, comprises two cannulas 46. Via the one cannula 46 a connection of the first connection means 24 to the connection point B at the left ventricle 14 is implemented, and via the other cannula 46 a connection of the second connection means 26 to the connection point A at the aorta 18 is implemented.

A heart support system 12 with a system unit 20 of such a simple structure, too, enables to relieve an insufficient heart 10 in such a way that, on the one hand the blood requirement of the body can be provided by the support system, and on the other hand an inherent additional load on the heart 10 is avoided or at least significantly suppressed by the heart support system 12.

Hereinafter the, structure, function and advantages of the heart support system 12 will be described once more in other words:

The entirety of the system 12 consists of an electromechanical apparatus, which is formed by one or more system units 20, 56, and a controller comprising a control and/or regulating means 48 and a corresponding measuring technique (pressure sensors 50, 52, 54) which controls the electromechanical apparatus in interaction with the cardiovascular system (as a control path of a control loop).

The electromechanical apparatus consists of the arrangement 22 of hydraulic pumps 30, 32, 34 and reservoirs 36 for blood, conventional cannulas 46 or optionally novel blocking cannulas 38 for connection to the cardiovascular system at various points, as well as corresponding connecting tubes.

With respect to the exact configuration of the arrangement of the elements of the electromechanical apparatus 20, 56 different embodiments are conceivable depending on the use of the blocking cannulas 38 and the number of connection points to the body. The blocking cannulas 38 can be introduced into the blood vessels 18 immediately behind the discharge valves of the heart (pulmonary artery or aorta 18) or in the large vessels in front of the cardiac atria (pulmonary vein or one of the vena cava). The blocking cannulas 38 aim at a hydraulic decoupling between the heart 10 and the subsequent circulatory system. The hydraulic decoupling by the blocking cannulas 38 is achieved by blocking the vessel, as well as by bypassing the blood discharged by the heart 10 from the body into the apparatus 20, 56. At the same time they enable the perfusion of the circulatory system by introducing the blood stream provided from the apparatus 20, 56 into the body.

The structural design of the blocking cannulas 38 is conceivable both as permanently blocked, as well as comprising a mechanism for temporary, reversible blocking of the cannula (such as by means of a balloon orflap). However, instead of the novel blocking cannulas, conventional cannulas can be used. Conventional cannulas 46 have a hose connection option, the blocking cannulas 38 have two hose connection options.

In the apparatus pumps 30, 32, 34, 58 driven by electric motors for delivering the blood are used.

A characteristic feature of the system 12, according to an embodiment, is a highly dynamic operation of the pumps 30, 32, 34, which means that the rotational speeds of the pumps 30, 32, 34 and, accordingly, the delivered blood flow can be changed sigificantly within one cardiac cycle (heart beat).

Accordingly, appropriately sized conventional rotary positive displacement pumps, in particular rotating piston pumps such as rotary piston pumps, gear pumps or flow pumps such as centrifugal pumps can be used.

The pumps 30, 32, 34 deliver the blood in one or both directions, that is, both from the connection point A, B, C, D to the reservoir 36 and vice versa.

If pump designs are used which due to the design only allow one delivery direction, for example flow pumps, a delivery in both directions can be achieved by use of two such pumps in conjunction with valves or non-return flaps. If the pumps 30, 32, 34 used do not block the fluid flow at standstill due to design, additional, for example electrically controlled, valves or non-return flaps can be used. Pumps 30, 32, 34, 58 implantable into the body and extracorporeal pumps may be used.

The reservoirs 36 collect the blood delivered out of the body by the pumps 30, 32, 34 at the connection points A, B, C, D. At the same time, the reservoirs 36 provide the volume (blood or blood substitute previously delivered out of the body) for delivery into the body. The reservoirs 36 are containers made of glass or plastic, which have corresponding connection options to the pumps 30, 32, 34, 58. Moreover, flexible plastic pouches can be used. If oxygen-rich and oxygen-poor blood is delivered at the selected connection points A, B, C, D two separate reservoirs 36 can be used for this purpose. However, a common reservoir 36 for all connection points is possible, too. If two reservoirs 36 are used, then by means of the pump 58 between the two reservoirs 36 a transfer of oxygen-rich blood into the oxygen-poor circulation and vice versa can be enabled. The reservoirs 36, like the pumps 30, 32, 34, 58, may be implemented extracorporeal or implantable into the body.

For connecting the components with each other hoses made of plastic can be used. The hoses can be extracorporeal or intracorporal. In addition, however, a constructive implementation of the connection is conceivable, in which connection hoses are omitted, for example, pumps 30, 32, 34 with an attached cannula.

However, a characteristic feature of the heart support system 12, according to an embodiment, is that at least two connection points A, B, C, D on one side of the heart and thus at least two pumps 30, 32 (or at least three pumps 30, 32, 34, if a completely blocking cannula is used) and at least one reservoir 36 are used.

The control and/or regulating means 48 consists of an electrical circuit and calculates and generates the control signals for all pumps 30, 32, 34 used. It also comprises means for operation by the user.

The calculation takes place taking into account measurements, which are obtained by appropriate sensors and measuring technology. To the measured state variables may belong:

Intravascular or ventricular/arterial blood pressures measured at the connection points. The pressure measurement may be achieved by conventional invasive pressure measuring catheters or by cannulas with integrated sensors 50, 52, 54.

The ventricular volume measured, for example, by conventional conductance catheters or by imaging techniques such as echocardiography.

Volume flow through the blood vessels leading to and originating from the heart 10—for example measured by means of ultrasound probes—and through the individual pumps 30, 32, 34 of the arrangement, in particular when no direct relationship between the pump speed and the volume flow exists.

The amount of blood in the reservoir 36, for example by weight measurement. The heart support system 12 causes an intervention on the otherwise naturally occurring blood flow into the heart 10 and out of the heart 10. This intervention consists in a redistribution of the blood.

The reduction of the preload is achieved by discharging the blood from the connection points of the vessels in front of the heart 10 as well as from the ventricle, in particular during the filling phase, into the reservoir 36. An increase in the preload is analogously achieved by introducing volume (blood or blood substitute fluid) from the reservoir 36 into the same connection points.

The reduction of the afterload is achieved by the discharge of blood from the connection points of the heart outlet vessels and from the ventricle, in particular in the contraction phase into the reservoir 36. An increase in the afterload is achieved by the introduction of volume from the reservoir 36 into the same connection points.

A characteristic feature is further, according to an embodiment, that these redistribution flows of the blood are not constant, but are temporally variable within the heart cycle. In this embodiment, objects are:

(a) primary: the precise adjustment (reduction or increase) of the preload and afterload; (b) generalized: the selective influence of the pressure and volume characteristics in the ventricle within the heart cycle and (c) by the way: the selective influence of the perfusion of the circulations (pressures and flows).

The user of the system 12 can select the specific targets and exact target parameters. The control and/or regulating means 48 calculates the redistribution flows and pump control signals necessary for compliance and outputs these to the pumps 30, 32, 34.

### LIST OF REFERENCE SYMBOLS

10 heart

12 heart support system

14 ventricle (blood vessel)

16 atrium (blood vessel)

18 aorta (blood vessel)

20 system unit

22 pump arrangement

24 first connection means

26 second connection means

28 third connection means

30 first pump

32 second pump

34 third pump

36 intermediate reservoir

38 cannula arrangement

40 cannula

42 cannula

44 blocking element

46 cannula

48 control and/or regulating means

50 first pressure sensor

52 second pressure sensor

54 third pressure sensor

56 further system unit

58 further pump

A, B, C, D connection points

1. Heart support system with at least one system unit comprising a pump arrangement, a first connection means connected to the pump arrangement and a second connection means connected to the pump arrangement, wherein the pump arrangement comprises a first pump, which is fluidically interconnected in a first flow path between the first and the second connection means, wherein
the pump arrangement further comprises an intermediate reservoir and a second pump, which together with the first pump are connected in a series connection in the first flow path, wherein the intermediate reservoir is fluidically arranged between the first pump and the second pump.

2. Heart support system according to claim 1, wherein the system unit comprises a third connection means connected to the pump arrangement, which is fluidically connected to the intermediate reservoir in such a way that the second pump and the intermediate reservoir are interconnected in a series connection in a second flow path between the second and the third connection means.

3. Heart support system according to claim 2, wherein the system unit comprises a cannula arrangement for such a connection of the second and the third connection means to a blood vessel, the blood flow directly through this blood vessel is completely or at least partially blocked at one point and is bypassed via the second flow path with the series connection of intermediate reservoir and second pump.

4. Heart support system according to claim 2, wherein in the second flow path between the second and the third connection means a third pump is connected such that a series connection of the second and the third pump and the intermediate reservoir fluidically interconnected between these pumps is achieved.

5. Heart support system according to claim 1, wherein the system unit comprises at least one cannula for a connection of the first connection means and/or the second connection means to a corresponding blood vessel.

6. Heart support system according to claim 2, wherein the first connection means and the third connection means are connected at the suction side to the pump arrangement and that the second connection means is connected at the pressure side to the pump arrangement.

7. Heart support system according to claim 2, wherein the at least one system unit is two system units.

8. Heart support system according to claim 7, further comprising a third pump which is fluidically interconnected between the intermediate reservoir of the two system units.

9. Heart support system according to claim 1, further comprising a control and/or regulating means for controlling the first and second pump.

10. Heart support system according to claim 1, further comprising pressure sensors for measuring the pressure in blood vessels and/or further comprising flow sensors for measuring the flow velocities of blood in blood vessels and/or further comprising sensors for measuring the filling volume of ventricles.

11. Cannula arrangement for a heart support system according to claim 2, which is adapted for such connection of the second and the third connection means to a blood vessel that blood flow directly through this blood vessel is blocked completely or at least partially at one point and is bypassed via the second flow path with the series connection of the intermediate reservoir and the second pump.

12. Cannula arrangement according to claim 11, further comprising a blocking element for completely blocking the direct blood flow through the blood vessel.

13. Use of a heart support system according to claim 1 for the therapeutic treatment of a heart and/or for the scientific examination of a heart.

14. Heart support system according to claim 4, further comprising a control and/or regulating means for controlling the third pump.
